(19) United States
(12) Patent Application Publication
(10) Pub. No.: US 2002/0150887 A1
(43) Pub. Date: Oct. 17, 2002

(54) Methods and nucleic acid probes for molecular genetic analysis of polluted environments and environmental samples

(75) Inventors: Akihiko Maruyama, Ibaraki (JP); Takanori Higashihara, Ibaraki (JP); Keiko Kitamura, Ibaraki (JP); Michinari Sunamura, Chiba (JP); Ryuichiro Kurane, Chiba (JP)

Correspondence Address: FISH RICHARDSON P.C., Suite 500, 4350 La Jolla Village Drive, San Diego, CA, 92122, US

(73) Assignee: National Institute of Advanced Industrial Science and Technology, Tokyo (JP)

(21) Appl. No.: 10/007,725

(22) Filed: Nov. 9, 2001

(30) Foreign Application Priority Data
Nov. 9, 2000 (JP) 341765/2000

(51) Int. Cl. C12Q 1/70; C12Q 1/68

(52) U.S. Cl. 435/5; 435/6

(57) ABSTRACT

The present invention provides novel methods for analyzing a dominant level of a certain microorganism with a specific function in an environment by detecting and quantifying nucleic acids of the microorganism by a non-RI method. Novel nucleic acid probes and an environmental diagnostic kit comprising a non-RI-labeled probe are also provided. Methods, probes and kits for analyzing and diagnosing a polluted or contaminated environment, e.g., an oil-polluted environment, is provided.

### RELATED APPLICATIONS

This application claims the benefit of foreign priority under 35 U.S.C. §119 to Japan Patent Application No. 341765/2000, filed Nov. 09, 2000. The aforementioned application is explicitly incorporated herein by reference in its entirety and for all purposes.

### TECHNICAL FIELD

The present invention generally relates to a diagnostic method for polluted or contaminated environments. Specifically, the present invention relates to a method for molecular genetic analysis and evaluation of environments polluted or contaminated by noxious chemicals, particularly petroleum and/or petroleum components, and to bioremediation processes of the polluted or contaminated environments by microorganisms. The present invention also relates to a method for molecular genetic detection and quantification of the microorganisms with specific functions, including microorganisms producing useful substances and harmful microorganisms such as pathogenic microorganisms contained in a natural or artificial environment. In addition, the present invention relates to a method for analyzing and evaluating utility and harm of the sample collected from an environment, wherein the method comprises analyzing a dominant level of a specific microorganism producing a useful substance including an enzyme, or a specific harmful microorganism such as a pathogenic microorganism from a natural or artificial environments.

### BACKGROUND

Qualitative and quantitative analysis of noxious chemicals in the environment using analytical instruments that combine gas chromatography or high-performance liquid chromatography with various types of elemental analysis, mass spectrometry, or spectrophotometry and others have been widely used to date as diagnostic methods for polluted environments. The COD and BOD methods permit measurement of the oxygen demand of polluted environmental samples. However, a simple and reliable molecular-genetic diagnostic method that targets at specific microorganisms, or groups of microorganisms, that live in polluted environments has not yet been developed. Various treatment techniques and technologies for some environmental pollutants, such as petroleum components, are also being developed through elucidation of the physico-chemical factors, which affect their natural cleanup, or “self-purification.”

However, there is no accurate method for analyzing and evaluating the constitution and fluctuation of the microbial population in the natural world that bears the burden of natural cleanup. Thus, it is currently difficult to evaluate the efficacy of treatment means and their universality.

A plate culture and counting technique with agar media and an “MPN,” or “most probable number,” culture and counting technique with liquid medium are used for environmental diagnosis that focuses on microorganisms in a polluted environment. However, these conventional culture and counting techniques have the drawback of requiring significant labor time and effort, and a long culture time for detection of specific microorganisms of interest.

In addition, very few of the microorganisms that live in the natural environment can be detected by these conventional isolation and cultivation techniques. Specifically, the percentage of microorganisms that can be isolated and cultured with such techniques is believed to be no more 1% in comparison to the total number of microorganisms obtained through a direct microscopic counting (see, e.g., Amann (1995): Phylogenetic Identification and In-Situ Detection of Individual Microorganism Cells without Cultivation, Microorganism. Rev. 59:143-169). The direct microscopic counting comprises staining the DNA of microorganisms with a fluorescent dye and counting them with an epifluorescence microscope; see, e.g., Hobbie (1977) Appl. Environ. Microbiol. 33:1225-1228; Porter (1980) Limnol. Oceanogr. 25: 943-948. Therefore, difficulty in analysis of the population structure and fluctuations of the microbial community, which live in the environment, and of the behavior of specific microorganisms, becomes a major obstacle.

Recently, molecular-genetic detection and quantification using DNA probes specific for the total microorganisms and a certain microorganism, which do not depend on the conventional isolation and cultivation techniques have therefore been attempted. For example, a method for detection and quantification at the molecular level by blot hybridization with radioisotope (RI)-labeled oligonucleotide probes has been reported by Giovannoni (1990) Nature 345:60-63. A probe-wash-off (dissociation) curve analytical technique with an RI-labeled probe, which is necessary for hybridization, has also been reported by Zheng (1996) Appl. Environ. Microbiol. 62:4504-4513.

However, since the conventional methods need the use of an RI-labeled probe, a special RI handling facility is essential for the detection and quantification thereof. This makes it difficult to perform monitoring in general laboratories and to develop the kits for the monitoring and the technique toward automation.

Another molecular-genetic technique for detection and quantification is quantitative PCR, which permits quantitative analysis by using DNA primers specific for the specific microorganism and amplifying the target nucleic acid region thereof (see, e.g., Lee (1993) Nuc. Acids Res. 21:3761-3766). However, by this technique it is difficult to obtain quantitative values on total microbial population in the environment and impossible to analyze the percentage (dominant level) of specific microorganisms versus total microorganisms, which poses a major technical limitation.

FISH technique is used for detecting specific microorganisms at the cellular level without isolation and cultivation (see, e.g., Amann (1995) ibid.). FISH-DC is effective for analysis of the percentage of specific microorganisms in the total microbial population at the cellular level in aquatic environmental samples (see, e.g., Maruyama (2000) Simultaneous direct counting of total and specific microorganism cells in seawater, using a deep-sea microorganism as biomarker, Applied and Environmental Microbiology, 66:2211-2215). However, some means for signal amplification is additionally required to detect and distinguish the many microorganism cells with low metabolic activity existing in environments. This currently remains a major technical limitation (Maruyama (2000) ibid.).

The diversity of microorganisms and the microbial population structure in a natural environment are gradually being elucidated by a series of techniques consisting of PCR, cloning, nucleotide sequencing analysis, and molecular phylogenic analysis using nucleic acids extracted directly from environmental microorganism samples (see e.g., Schmidt (1991) Analysis of a marine picoplankton community by 16S rRNA gene cloning and sequencing, J. Bacteriol., 14:4371-4378) in addition to conventional isolation and cultivation techniques.

The appearance of certain microorganism groups in environments polluted by petroleum, etc. that were difficult to be isolated and cultivated by conventional techniques has being clarified by these methods. For example, Cycloclasticus pugetii, a bacterium that degrades aromatic hydrocarbons, has been found on the west coast of the United States (Dyksterhouse (1995) Cycloclasticus pugetii gen. nov., sp. nov., an aromatic hydrocarbon-degrading bacterium from marine sediments, Int. J. Syst. Bacteriol., 45:116-123) and the Japan Sea coast, and Alcanivorax borkumensis, a bacterium that degrades aliphatic hydrocarbons, has been found in the North Sea (Yakimov (1998): Alcanivorax borkumensis gen. nov., sp. nov., a new hydrocarbon-degrading and surfactant-producing marine bacterium, Int. J. Syst. Bacteriol., 48:339-348; the Seto Inland Sea coast, and the Japan Sea coast (Kishiyoshi and Harayama: Seibutsu Kogakkai, collective abstracts, p. 291, 1998). Dominant level analysis of Cycloclasticus pugetii among the above has already been performed on the cellular level by a method with improved accuracy that combines the aforementioned direct count method and MPN method (Japanese Patent Application No. 11-237818). However, there are no examples of dominant level analysis of these microorganisms being performed with molecular-genetic techniques, regardless of whether or not RI is used.

### SUMMARY

The present invention provides a novel methods for the molecular-genetic detection and quantification of both total microorganisms that live in a natural environment and specific microorganisms therein. In one aspect, the methods of the invention can be performed without utilizing radioisotopes; i.e., it is a non-radioisotope (RI) method. In one aspect, the methods of the invention can be performed without using a polymerase chain reaction (PCR) technique.

Another aspect of the present invention provides methods for diagnosing polluted or contaminated environments using the aforementioned method to monitor environments polluted or contaminated by chemicals, such as those found in oil and petroleum, and to molecular-genetically analyze and evaluate the bioremediation process of the polluted or contaminated environments by microorganisms.

Another aspect of the present invention provides methods for molecular genetic detection and quantification of microorganisms with specific functions, including microorganisms producing useful substances and harmful microorganisms such as pathogenic microorganisms contained in a natural or artificial environment.

Another aspect of the present invention provides methods for molecular genetic analyzing and evaluating utility and harm of the sample collected from an environment, wherein the method comprises analyzing a dominant level of a specific microorganism producing a useful substance, or a specific harmful microorganism such as the pathogenic microorganism from a natural or artificial environments.

Another aspect of the present invention provides novel nucleic acid probes specific for Alcanivorax borkumensis, a petroleum-degrading bacterium, which is useful in all the methods.

Environmental pollutants such as petroleum that are effluxed from an artificially controlled environment into a outside environment are gradually degraded by microbial activities in the natural environment and naturally cleansed over time. Degradation requires a long time in the case of non-labile substances, which are recalcitrant to biodegradation such as PCBs. However, when this pollution affects even the growth of common microorganisms, specific microorganisms (groups) appear predominantly in these polluted or contaminated environments.

For example, it is known that hydrocarbon-degrading bacteria are widely distributed at a level of no more than 1% of the total microbial population at a given site in the ocean, while their percentage frequently exceeds 10% in parts of the ocean polluted by petroleum (see, e.g., Atlas (1995): Petroleum biodegradation and oil spill bioremediation, Marine Pollution Bulletin, 31, 178-182; Atlas (1992): Hydrocarbon Biodegradation and Oil Spill Bioremediation, In: Advances in Microbiological Ecology, Ed. K. C. Marshall, Plenum Press, New York, 12, 287-338). The percentage of petroleum-degrading bacteria versus the total microbial population appears to reflect the degree of petroleum pollution (Atlas (1992) ibid.).

The number of polycyclic aromatic hydrocarbons [PAHs]-degrading bacteria in the sediment of harbors polluted by creosote (containing approximately 85% PAHs) drained from wood-treatment facilities has also being surveyed (Geiselbrecht (1996): Enumeration and Phylogenetic Analysis of Polycyclic Aromatic Hydrocarbon-Degrading Marine Bacteria from Puget Sound Sediment, Appl. Environ. Microbiol., 62:3344-3349). According to this survey, the number of PAHs-degrading bacteria enumerated by MPN method was approximately 104 to 107 MPN/g (dry weight) at polluted sites and approximately 103 to 104 MPN/g (dry weight) at non-polluted sites. However, the total number of bacteria in the sediment showed almost no difference between polluted and nonpolluted sites as being about 109/g (dry weight) at both sites. In other words, not only were PAHs-degrading bacteria 10 to 1000-fold greater in count at creosote-polluted sites than at non-polluted sites, but the percentage relative to the total number of bacteria also increased to around 1%. Thus, the percentage of pollutant-degrading bacterial groups versus the total microbial population is found to increase in reflection of the pollution of the environment. The dominant level of pollutant-degrading bacteria thus serves as a good indicator of a polluted environment.

However, all the numbers of the petroleum-degrading bacteria and PAHs-degrading bacteria in the aforementioned reports were determined by plate count methods and MPN methods, both of which require significant time and effort. As mentioned above, since the number of microorganisms that can be isolated and cultivated is usually no more than 1% of the total microbial population, the enumeration by the culture methods may not adequately reflect the number of pollutant-degrading microorganisms in the environmental microbial population. Furthermore, specific pollutant-degrading bacteria (which belong to specific genera and species or have genetic information that can be specified by means of molecular phylogenetic classification) in the microbial population in the environment are not enumerated or quantified as in the method of the present invention.

Therefore, if it were possible to simply and rapidly monitor the behavior of specific microorganisms including these degrading bacteria as being environmental indicators and the percentage thereof in the total microbial population (dominant level), it would be possible to provide accurate and timesaving diagnosis of the polluted environment such as the extent of pollution, and the degree of repair and recovery of the polluted environment. In order to analyze the total microbial population and microorganisms with specific functions, development of detection and quantification techniques are required in the art, which can be substituted for the conventional isolation and cultivation methods with a severe technical limit in their availability, and can be used in a general manner for diagnosis of polluted environment. The inventors meet the requirement in the art through the following steps.

First of all, two types of petroleum-degrading bacteria with different degradation properties, aliphatic hydrocarbon-degrading bacteria and aromatic hydrocarbon-degrading bacteria, were selected. These are being demonstrated to appear dominantly in oil-polluted environments. Nucleotide probes specific for each bacterium are developed.

The selected aliphatic hydrocarbon-degrading bacteria degrade aliphatic hydrocarbons but not degrade aromatic hydrocarbons and sugars such as glucose. On the other hand, the selected aromatic hydrocarbon-degrading bacteria degrade recalcitrant polycyclic aromatic hydrocarbons but not utilize aliphatic hydrocarbons and sugars such as glucose. Therefore, the behaviors of these two types of petroleum-degrading bacteria are believed to fluctuate depending on the hydrocarbon components and their concentrations in the spilled oil. Being able to quantitatively detect the nucleic acid of these two petroleum-degrading bacteria present in oil-polluted environments and analyze the microbial population at the molecular genetic level should make it possible to analyze and evaluate the process of self-purification of pollutants and of the bioremediation of the polluted environment through such as the percentage, concentration, and rise and fall of hydrocarbon components in the environment.

The inventors first succeeded in developing a non-RI method for molecular genetic detection and quantification of overall microorganisms and specific microorganisms in an environment. Specifically, they succeeded in developing a relative molecular quantification method for microorganism nucleic acid in the environment by a non-RI technique. In the present method, the determination of the condition of hybridization, which is one step in using a novel probe, could be performed without radioisotopes. According to the method, for hybridization and detection steps, fluorescence- or chemiluminescence-labeled probes are utilized and detected by these labels. Thus no radioisotope is required throughout the entire steps in this method.

It is very difficult to accurately estimate the efficiency of the extraction of nucleic acid from the microorganisms in environmental samples. Because various contaminants are comprised in environmental samples and it is difficult to set internal standards adequately, in addition, nucleic acids, particularly RNA, are susceptible to enzymatic degradation. This imposes a severe limitation on accurately quantifying (absolute quantification) the nucleic acid content present in the original sample, even when a highly purified nucleic acid sample is prepared and quantified. Consequently, the most effective method is believed relative evaluation by selecting the target sequence and the standard sequence from nucleic acid molecules that demonstrate identical behavior during extraction and purification, which may be identical or multiple molecules, and determining the ratio thereof from the amounts thereof. This analytical technique is referred to as the relative molecular quantification technique.

The principle of this relative molecular quantification technique is as follows. Assuming multiple hybridizations in the same sample, the correct relative value is determined by correcting the hybridization value of the probe (which can hybridize with the target sequence) for specific microorganism using the hybridization value of the universal probe (which can hybridize with the standard sequence) for all living organisms. In actuality, normalization is provided by using the mean of the values determined in various standard strains as reference data. Although the methods disclosed as conventional RI methods can be employed in this normalization, the novelty lies in for the first time doing the entire process by a non-RI method. Furthermore, major characteristic and advantage of this analytical technique is that it does not use PCR procedure.

The concentrated and cryopreserved microorganism samples, which have been collected at the polluted site immediately after an oil spill accident in 1997 were analyzed using this newly developed technique. As a result, the dominant level of aliphatic hydrocarbon-degrading bacteria and aromatic hydrocarbon-degrading bacteria described above among the total microbial population could be estimated. The newly developed nucleic acid probe specific for the aliphatic hydrocarbon-degrading bacterium permitted detection and quantification at the cellular level by fluorescent in situ hybridization method (FISH).

As stated above, the non-RI method of the present invention is effective in the molecular-genetic diagnosis of actual petroleum-polluted environments and in the analysis and evaluation of the bioremediation process. The developments culminated in the present invention, because this technique is very convenient for making diagnostic kits and designing automation and also appears extremely advantageous for the diagnosis of environments contaminated by chemicals other than petroleum as well as microbes and in the analysis and evaluation of bioremediation thereof. The present invention was attained based on these findings.

In summary, the present invention includes the following aspects:

[1] In one aspect, a method for analyzing a dominant level of a certain microorganism with a specific function in an environment by detecting and quantifying nucleic acids of the microorganism with a specific function from a natural or artificial environment by a non-RI method, comprising the following steps of:

1) extracting, purifying, and preserving a target nucleic acid from a microorganisms-containing sample collected from the natural or artificial environment;

2) selecting a specific nucleic acid sequence from sequence information of the target nucleic acid of the microorganism with a specific function, and synthesizing a nucleic acid with the selected sequence, and labeling the synthesized nucleic acid with a non-RI-label to prepare a non-RI-labeled nucleic acid probe specific for the microorganism;

3) immobilizing the target nucleic acid extracted and purified in step 1) to a substrate for hybridization, and adding the non-RI-labeled nucleic acid probe specific for the microorganism, followed by hybridization and washing;

4) obtaining an image of the signals derived from the non-RI label on the hybridized nucleic acid probe, and determining from the image, quantified values of the target nucleic acid from the microorganism; and

5) calculating a dominant level of the microorganism through comparing the quantified values of the microorganism in step 4) with those determined for all organisms or any of domains in the living world.

[2] In one aspect, the washing of step 3) is performed at an optimum washing temperature which is determined from the probe-wash-off curve for the non-RI-labeled nucleic acid probe specific for the microorganism with a specific function obtained by a non-RI method.

[3] In one aspect, the non-RI-labeled nucleic acid probe is labeled with a fluorescent or chemiluminescent label.

[4] In one aspect of the methods, the microorganism with a specific function is a microorganism that degrades a specific chemical.

[5] In one aspect of the methods, the specific chemical is a noxious chemical.

[6] In one aspect of the methods, the specific chemical is a petroleum or a petroleum component.

[7] In one aspect of the methods, the microorganism with a specific function is a microorganism producing a useful substance.

[8] In one aspect of the methods, the microorganism with a specific function is a harmful microorganism including a pathogenic microbe.

[9] The invention provides a method for evaluating and diagnosing a function of the microbial population in an environment, wherein the method comprises analyzing a dominant level of a certain microorganism with a specific function in a natural or artificial environment using a method of the invention, such as those as set forth above in [1]-[6].

[10] The invention provides a method for analyzing and diagnosing a polluted or contaminated environment using a method of the invention, such as those as set forth above in [1]-[6].

[11] The invention provides a method for analyzing and diagnosing an environment polluted or contaminated by a noxious chemical using a method of the invention, such as those as set forth above in [4] or [5].

[12] The invention provides a method for analyzing and diagnosing an oil-polluted or contaminated environment using a method of the invention, such as those as set forth above in [6].

[13] The invention provides a DNA or an RNA probe with length of from between about 10 to about 50 bases, or, about 15 to about 40 bases, or, about 20 to about 30 bases, or, about 15 to about 25 bases, comprising, or, consisting essentially of, or, consisting of, any part of the nucleotide sequence as set forth in SEQ ID NO:5 or a corresponding RNA sequence thereto, the probe being capable of hybridizing specifically with a nucleic acid derived from any of petroleum-degrading bacteria belonging to the phylogenetic group or genus of Alcanivorax.

[14] The invention provides a DNA or an RNA probe comprising, or, consisting essentially of, or, consisting of, the nucleotide sequence as set forth in SEQ ID NO:1 or a corresponding RNA sequence thereto, which enables detection or quantification of a petroleum-degrading bacterium by hybridizing specifically with a nucleic acid derived from any petroleum-degrading bacteria belonging to the phylogenetic group or genus of Alcanivorax.

[15] The invention provides a DNA or an RNA probe, such as those set forth in [13] or [14], above, wherein the petroleum-degrading bacteria belonging to the phylogenetic group or genus of Alcanivorax are Alcanivorax borkumensis or its closely related species.

[16] The invention provides a method for analyzing and diagnosing an oil-polluted or contaminated environment, wherein the method comprises analyzing a dominant level of a petroleum-degrading bacterium belonging to the phylogenetic group or genus of Alcanivorax and/or to the genus of Cycloclasticus in microbial population in the environment by a method of the invention, e.g., the method of [1], using at least one probe selected from the group consisting of a DNA or an RNA probe of [13] or DNA or RNA probes comprising, or, consisting essentially of, or, consisting of, the nucleotide sequence as set forth in SEQ ID NO:1 or a corresponding ribonucleotide sequence thereto. In one aspect, these probes are capable of hybridizing specifically with nucleic acids derived from any of petroleum-degrading bacteria belonging to the phylogenetic group or genus of Alcanivorax, and DNA or RNA probes with length of from between about 10 to about 50 bases, or, about 15 to about 40 bases, or, about 20 to about 30 bases, or, about 15 to about 25 bases, comprising, or, consisting essentially of, or, consisting of, any part of the nucleotide sequence as set forth in SEQ ID NO:6 or a corresponding ribonucleotide sequence thereto, and DNA probes comprising sequences as set forth in SEQ ID NOs:2-4 or corresponding RNA probes thereto, which probes are capable of hybridizing specifically with nucleic acids derived from any of petroleum-degrading bacteria belonging to the genus Cycloclasticus, as the non-RI-labeled nucleic acid probe specific for a certain microorganism with a specific function.

[17] In one aspect, as in the method of [16], the methods are characterized by analyzing a dominant level of any microorganism selected from the group consisting of an aliphatic hydrocarbon-degrading bacterium, Alcanivorax borkumensis, or its closely related species and/or an aromatic hydrocarbon-degrading bacterium, Cycloclasticus pugetii, or its closely related species in the microbial population in an environment.

[18] The invention provides a environmental diagnostic kit comprising a non-RI-labeled probe, wherein the probe is prepared by non-RI-labeling a nucleic acid probe selected from the group consisting of a DNA or an RNA probe of the invention, such as those described in [13], an DNA or RNA probes with length of from about 10 to about 50 bases, or, about 15 to about 40 bases, or, about 20 to about 30 bases, or, about 15 to about 25 bases, comprising, or, consisting essentially of, or, consisting of, any part of the nucleotide sequence as set forth in SEQ of ID NO:6 or a corresponding ribonucleotide sequence thereto which is capable of hybridizing specifically with a nucleic acid derived from a petroleum-degrading bacterium belonging to the genus Cycloclasticus, and a DNA or an RNA probe that comprise, or, consist essentially of, or, consist of, a nucleotide sequences as set forth in SEQ ID NOs:1-4 or corresponding ribonucleotide sequences thereto.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### DESCRIPTION OF DRAWINGS

FIG. 1 schematically shows data from the wash-off curve of the Albo 222 probe for the detection of Alcanivorax borkumensis in extracted rRNA-DNA probe hybridization, as discussed in detail in the Examples, below.

FIG. 2 schematically shows data from the wash-off curve of the Cypug 829 probe for the detection of Cycloclasticus pugetii in extracted rRNA-DNA probe hybridization, as discussed in detail in the Examples, below.

Like reference symbols in the various drawings indicate like elements.

### DETAILED DESCRIPTION

The present invention provides methods for analyzing a dominant level of a certain microorganism with a specific function in an environment by detecting and quantifying nucleic acids of the microorganism with a specific function from a natural or artificial environment by a non-radioisotope (RI) method, comprising the following steps of: 1) extracting, purifying, and preserving a target nucleic acid from a microorganisms-containing sample collected from the natural or artificial environment; 2) selecting a specific nucleic acid sequence from sequence information such as 16S rDNA of the target nucleic acid of the microorganism with a specific function, and synthesizing a nucleic acid with the selected sequence, and labeling the synthesized nucleic acid with a non-RI-label to prepare a non-RI-labeled nucleic acid probe specific for the microorganism; 3) immobilizing the target nucleic acid extracted and purified in step 1) to a substrate for hybridization, and adding the non-RI-labeled nucleic acid probe specific for the microorganism, followed by hybridization and washing; 4) obtaining an image of the signals derived from the non-RI label on the hybridized nucleic acid probe, and determining from the image, the quantified values of the target nucleic acid from the microorganism; and 5) calculating a dominant level of the microorganism through comparing the quantified values of the microorganism in step 4) with those determined for all organisms or any of domains in the living world.

In this method, measurement of the amount of all organisms and the population levels of each domain in the living world is indispensable for understanding the magnitude of the total microbial population (i.e. the size of the population) in the environmental sample of interest, and is extremely important in the step that estimates the dominant level of a microorganism with a specific function therein.

The term “dominant level” used herein refers to the ratio of a certain microorganism of interest to the total microbial population in a sample.

As used herein, the term “natural environment” means the biosphere on the earth, including all of the environments on the earth in which microorganisms live, such as hydrosphere such as oceans, lakes, and rivers, geosphere such as the soil, on and under the land, under the ocean floor, atmosphere such as the surface layer of the earth, and plant and animal bodies and carcasses.

The term “artificial environment” means an environment artificially controlled to different degrees, including for example, the environment inside a laboratory flask or experimental apparatus, the environment inside production tanks such as fermentation tanks used in the chemical and bio-industrial fields, such as food production, fermentation technology, and pharmaceutical industry, and environments associated with the artificial production, processing, storage, transportation, utilization, and discard of water such as city water supply, water used in households and industry, cooling water, circulating water, wastewater, and sewage. It means all artificial environments in which microorganisms may be present or contaminated.

The term “a microorganism with a specific function” means a microorganism has a function of which is specified by means such as culture or genetic analysis among microorganisms living in the natural environment. Examples include, but are not limited to, microorganisms that can degrade chemicals such as petroleum and petroleum components, noxious chemicals such as organochlorine compounds including trichloroethylene, PCB, and dioxin, endocrine disrupting materials (such as alkylphenols, bisphenol A, and phthalic acid esters), organomercury compounds, cyanogen compounds, and organotin compounds. Other examples are microorganisms producing useful substances, including various antibiotics and useful enzymes such as chitinases, lipases, cellulases, xylanases, and lignin-degrading enzymes, pathogenic microorganisms such as Escherichia coli including E. coli O157 strain, Vibrio cholerae, and Bacillus anthracis, and the harmful microorganism, such as sulfate-reducing bacteria which cause occurrence of corrosion of the tank and offensive odor by the generation of hydrogen sulfide in the tank, are also included in the microorganisms with specific functions.

The term “closely related species” used herein means the species in which 16S rDNA sequences have homology 90% or more, preferably 94% or more, most preferably 95% or more.

Examples of the closely related species of Alcanivorax borkumensis include, but are not limited to: Alcanivorax sp. ST-T1, Alcanivorax sp. Wf-1, Alcanivorax sp. Shm-2, Alcanivorax sp. K3-3, Alcanivorax sp. K2-1, Alcanivorax sp. TE-9; and, Fundibacter jadensis.

The closely related species of Cycloclasticus pugetii include, but are not limited to, for example Cycloclasticus sp. M4-6, Cycloclasticus sp. E16S, Cycloclasticus sp. W, Cycloclasticus sp. G, Cycloclasticus sp. N3-PA321, and Cycloclasticus sp.(Accession No. AF 148215 of 16SrDNA, DDB).

The word “microorganism-containing samples collected from a natural environment or artificial environment” means natural environmental samples such as sea water, lake water, river water, bottom mud, sediment, soil, minerals, underground water, pore water, and plants and animals, the aforementioned artificial environmental samples, and samples in which the microorganisms from the environment have been concentrated by means such as filtration and centrifugation when there are few microorganisms in these environments. For example, the microorganisms in environmental water can be concentrated on a filter by filtration using a filter such as a membrane filter or hollow-fiber membrane filter with a pore size of 0.2 μm, which is smaller than the cell size of many common microorganisms and the product of this procedure can be used as the sample. Alternatively, the sample water can be filtered by passing it horizontally rather than vertically using for example a tangential flow filter (Millipore, Bedford, Mass.) with a membrane filter with a pore size of 0.2 μm, and the resulting concentrated solution can be used as the sample. The microorganisms can also be precipitated and concentrated by subjecting the sample directly to high-speed centrifugation, e.g., by centrifuging for 10-100 min at approximately 8000 X g or more, and the resultant sample can also be used for nucleic acid extraction.

Known methods (e.g., methods described in Murray (1980) Nuc. Acids Res. 8:4321-4325) can be used for extracting the nucleic acid, DNA or RNA, from the aforementioned microorganism-containing samples. Purification techniques using hydroxyapatite (e.g., Purdy (1997): Use of 16S rRNA-targeted oligonucleotide probes to investigate the occurrence and selection of sulfate-reducing bacteria in response to nutrient addition to sediment slurry microcosms from a Japanese estuary, FEMS Microbiol. Ecol., 24, 221:234) are also advantageous in the case of samples such as soil and sediment. When the subject of analysis is RNA such as 16S rRNA in particular, a commercial available RNA extraction kit such as a Qiagen RNEASY KIT™, Stratagene RNA RT-PCR Miniprep kit, Clontech NUCLEOSPIN™ RNA kit, or Ambion RNAQUEOUS™ kit may be used. When the sample contains a large amount of contaminants, the efficiency of purification of the extracted nucleic acid can be improved by combining several of these nucleic acid extraction methods. The degree of purification can be confirmed easily by measuring the spectrum of absorbance near a wavelength of 220 to 400 nm by spectrophotometer and comparing it with that of pure RNA and DNA samples. In all cases, the careful attention is necessary to prevent contamination of biological materials such as DNase or RNase during the extraction procedure.

The word “preparing a non-RI-labeled nucleic acid probe” means to prepare by labeling a nucleic acid, e.g., an oligonucleotide, e.g., those having a length of as much as between about 10 to about 50 bases (but, the probe can be smaller or larger), that is selected as the probe so as to permit analysis by detection techniques without radioisotopes such as detection of fluorescence, chemiluminescence, bioluminescence or chemical fluorescence. Here, the oligonucleotide can be synthesized by known methods such as the phosphoramide method or triester method. It may also be synthesized with a DNA synthesizer.

The term “nucleic acid” as used herein refers to a deoxyribonucleotide or ribonucleotide in either single- or double-stranded form. The term encompasses nucleic acids containing known analogues of natural nucleotides. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). PNAs contain non-ionic backbones, such as N-(2-aminoethyl) glycine units. Phosphorothioate linkages are described, e.g., by U.S. Pat. Nos. 6,031,092; 6,001,982; 5,684,148; see also, WO 97/03211; WO 96/39154; Mata (1997) Toxicol. Appl. Pharmacol. 144:189-197. Other synthetic backbones encompassed by the term include methylphosphonate linkages or alternating methylphosphonate and phosphodiester linkages (see, e.g., U.S. Pat. No. 5,962,674; Strauss-Soukup (1997) Biochemistry 36:8692-8698), and benzylphosphonate linkages (see, e.g., U.S. Pat. No. 5,532,226; Samstag (1996) Antisense Nucleic Acid Drug Dev 6:153-156). The term nucleic acid is used interchangeably with gene, DNA, RNA, cDNA, mRNA, oligonucleotide primer, probe and amplification product.

The nucleic acid, e.g., oligonucleotide, may be labeled with a fluorescent dye or a peptidic tag such as an antigen for example digoxigenin. Examples of fluorescent dyes include Rhodamine, Fluorescein-isothiocyanate (FITC), Lucifer Yellow CH, Rhodamine 123, Pyronin Y, Propidium iodide, Ethidium homodimer, Carboxyfluorescein diacetate (CFDA), Fluorescein diacetate (FDA), Carboxyfluorescein diacetate-acetoxymethyl ester (CFDA-AM), 5-cyano-2,3-ditolyl tetrazolium chloride (CTC), Tetramethylrhodamine isothiocyanate (TRITC), Sulforhodamine 101 acid chloride (Texas Red), Cy3, Cy5, Cy7, and 2-hydroxy-3-naphthoic acid-2′-phenylanilide phosphate (HNPP). For labeling a probe with fluorescent dye, the biotin/avidin or digoxigenin/anti-digoxigenin antibody system may be employed. Alternatively, when the probe is labeled with a tag such as an antigen, the chemiluminescence or fluorescence (including chemical fluorescence) produced via enzymatic reaction of the substrate may be detected using enzyme-immunological means.

In any case, a substrate such as an organic-coated slide glass or membrane filter used in hybridization that has very weak background fluorescence and luminescence can preferably be used. A fluorescent dye or antigen-antibody-enzyme-substrate reaction system used for the present method preferably exhibits no or very little non-specific binding to the substrate. For example, a probe labeled with Cy 5 is useful when fluorescence is detected using a membrane filter as the hybridization substrate, and a probe labeled with digoxigenin (DIG) is useful for chemiluminescence detection, using alkaline phosphates-chemiluminescence substrate system that employs CDP-Star as the substrate (e.g., a kit made byRoche).

In “determining a wash-off curve of the probe by a non-RI method”, first hybridization of the extracted nucleic acid sample from microorganisms is performed with a DNA probe labeled with a fluorescent dye or a peptidic tag such as digoxigenin as described above on a hybridization substrate, e.g., a positively charged nylon membrane filter or slide glass organic-coated with a substance such as poly-L-lysine. Next, the fluorescence or chemiluminescence is measured in accordance with methods conducted using RI-labeled DNA probes by the method of Zheng et al. (Zheng (1966) ibid.). Specifically, the hybridized sample is washed with a fresh washing solution at each temperature raised at intervals of several degrees centigrade, and the fluorescence or chemiluminescence derived from the labeled DNA probe released into each wash solution is measured. Thus, the probe-wash-off curve can be determined by plotting the measured values versus the wash temperature. Finally, the temperature near the inflection point seen on the low temperature side in the sigmoid probe-wash-off curve is taken as the optimum wash temperature of the probe.

In “immobilization of the nucleic acid to the substrate for hybridization”, a set quantity of the nucleic acid sample is added dropwise and attached (blotted) to a substrate surface, e.g., positively charged nylon membrane filter or slide glass organic-coated with a substance such as poly-L-lysine. In the case that a filter is used as the substrate, the nucleic acid can be firmly attached or fixed to the filter by a UV apparatus, e.g., UV-radiation treatment using a UV crosslinker (Stratagene, San Diego, CA) or alkaline solution treatment. In the case that a slide glass is used as the substrate, the nucleic acid sample can be attached to coated surface of the glass formed by a substance that mediates the attachment between the substrate (such as poly-L-lysine) and nucleic acid. Any substrate surface or variation thereof can be used, e.g., including a “array” or “biochip” format, as described, e.g., in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049.

The terms “hybridizing specifically to” and “specific hybridization” and “selectively hybridize to,” as used herein refer to the binding, duplexing, or hybridizing of a nucleic acid molecule to a particular nucleotide sequence under stringent conditions. The term “stringent conditions” refers to conditions under which a probe will hybridize preferentially to its target subsequence, and to a lesser extent to, or not at all to, other sequences. A “stringent hybridization” and “stringent hybridization wash conditions” in the context of nucleic acid hybridization (e.g., as in array, Southern or Northern hybridizations) are sequence dependent, and are different under different environmental parameters. Stringent hybridization conditions that can be used to identify nucleic acids can include, but are not limited to, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Those of ordinary skill will readily recognize that alternative but comparable hybridization and wash conditions can be utilized to provide conditions of similar stringency. However, the selection of a hybridization format is not critical, as is known in the art, it is the stringency of the wash conditions that set forth the conditions which determine whether a soluble, sample nucleic acid will specifically hybridize to an immobilized nucleic acid. Wash conditions used to identify nucleic acids include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). see, e.g., Sambrook, ed., Molecular Cloning: A Laboratory Manual (2ND ED.), Vols. 1-3, Cold Spring Harbor Laboratory, (1989); Current Protocols in Molecular Biology, Ausubel, ed. John Wiley & Sons, Inc., New York (1997); Laboratory Techniques in Biochemistry and Molecular Biology: Hybridization with Nucleic Acid Probes, Part I. Theory and Nucleic Acid Preparation, Tijssen, ed. Elsevier, N.Y. (1993) for detailed descriptions of equivalent hybridization and wash conditions and for reagents and buffers, e.g., SSC buffers and equivalent reagents and conditions.

In “hybridization and washing by non-RI method”, the following procedure is performed. The substrate to which the nucleic acid sample has been immobilized by the above-mentioned method is rinsed for 30 minutes at an arbitrary temperature, e.g., 35° C., in a hybridization solution (e.g., the solution described by Raskin (1994): Group-Specific 16S rRNA Hybridization Probes to Describe Natural Communities of Methanogens, Applied and Environmental Microbiology, 60:1232-1240) (prehybridization process). Next, it is contacted overnight at an arbitrary temperature, e.g., 35° C., in the aforementioned hybridization solution containing the above non-RI-labeled DNA or RNA probe (hybridization process). The hybridization process is essential in the present method. Finally, it can be rinsed twice for 30 minutes each at the optimum wash temperature for the used probe in a wash solution (e.g., the solution described by Zheng (1996) ibid.) (wash process).

In “determination of the quantified values by acquiring and analyzing dot- or slot-blotted images derived from the hybridized non-RI-labeled nucleic acid probe”, the following procedure is performed. After hybridization and washing, the dot-or slot-blotted image of the probes which are still associated to the complementary nucleic acids on the substrate is acquired by an instrument that accords with the type of substance used to label the probe: a fluorescence image analyzer in the case of fluorescence, e.g., instruments such as FLUOROIMAGER™, STORM™, or TYPHOON™ (Amersham Pharmacia Biotech), FX PRE™ (BioRad), or an instrument such as a photo-multiplier, cooling CCD camera, or an instrument such as a cooling CCD camera or photon counting camera in the case of chemiluminescence.

The term “all organisms” means the sum total of all three domains of the living world comprising Archaea, Bacteria, and Eucarya. The word “calculate the dominant level of the microorganisms with specific functions versus all organisms and each domain of the living world” means calculation taking the sum total of the quantified values measured in each three of the aforementioned domains or the quantified value measured in the domain to which the microorganisms with a specific function belong as the denominator and the quantified value measured taking the microorganism with a specific function as the subject as the numerator. However, several corrections as described below are necessary in actual practice.

For example, taking 16S rRNA as the subject, the quantified values are not necessarily the same due to its tertiary structure even when the target nucleotide sequence regions of two types of probes are present in a 1:1 ratio in one molecule. Therefore, correction is necessary to make the values accurate.

Absolute quantification of specific target nucleotide sequence regions in the microorganism with a specific function is also impossible as long as the extraction and purification efficiency is not estimated accurately. Therefore, the amount of the target nucleotide sequence present has to be evaluated relatively by taking the nucleotide sequence region (universal region) common to all three of the aforementioned domains as the subject of comparison and determining the ratio of the target nucleotide sequence region to it.

In the case of rRNA, the amount present per cell is also believed to vary depending on the type as well as cell activity. Moreover, the ratio of the specific region in each domain to the aforementioned universal region is believed to differ depending on type even in the same domain due to differences in their conformation. Consequently, fluctuations in the quantified values due to the differences in type have to be corrected in calculation by determining the average value by measuring several representative standard strains beforehand.

Finally, the background of quantified values due to the hybridization substrate itself and the nonspecific binding of the DNA probe to the substrate has to be subtracted from the measured value.

Estimation of the dominant level of the microorganisms with specific functions can be corrected using the following formula 1 previously proposed in an RI technique (S. J. Giovannoni et al., 1990, ibid.).

$$\% X = \frac{δ C / δ N \times {( δ U / δ N )}^{- 1} - {\sum }_{i = 1}^{r} [ δ {R}_{i} / δ N \times {( δ U / δ N )}^{- 1} ] {r}^{- 1}}{{\sum }_{i = 1}^{p} [ δ {P}_{i} / δ N \times {( δ U / δ N )}^{- 1} ] {p}^{- 1} - {\sum }_{i = 1}^{r} [ δ {R}_{i} / δ N \times {( δ U / δ N )}^{- 1} ] {r}^{- 1}} \times 100$$

wherein;

X: an amount of specific RNA in an RNA sample (which corresponds to the dominant level of a specific microorganism);

δC/δN: the group-specific probes bound per unit of sample RNA;

δRi/δN: group-specific probes bound per unit of heterologous RNA standard;

δPi/δN: group-specific probes bound per unit of homologous RNA standard;

p: number of homologous RNA standard; and

δU/δN: universal probes bound per unit of RNA (either sample or standard).

DNA probes such as the Euba 338, Arch 915, and Euka 502 described by Amann et al. (Amann (1995) ibid.) can be used in the detection and determination of each domain of Bacteria, Archaea, and Eucarya respectively in this calculation. Probes such as Univ 1390 (Zheng (1996) ibid.) can be used as the DNA probe that targets the region common to all living organisms. Probes that have as the subject a nucleotide sequence region specific for the microorganism, e.g., a nucleotide sequence region specific for the species from among the genetic nucleotide sequence information such as 16S rDNA, 18S rDNA, 23S rDNA, and gyrB, are synthesized for the detection and determination of the microorganisms with specific functions. Non-RI-labeled nucleic acid probes can be produced by the method discussed above.

The function of microbial population in that environment can be evaluated by analyzing the dominant level of the microorganisms with specific functions that are predominant in the natural or artificial environment using the above method. As used herein, the term “function of microbial population” means the function performed as a whole by the multiple types of microorganisms living in the environment of interest. Here, the term species of microorganism is preferably the species that is an ordinary classification unit, but may be an experimental functional unit, e.g., an identical functional unit meaning a petroleum-degrading bacteria group or PCB-degrading bacteria group. Therefore, the function of only a single type of microorganism with a specific function is not called the function of microbial population.

For example, when the target is the degradation of petroleum, many microorganisms other than microorganisms that degrade aliphatic hydrocarbons and those that degrade aromatic hydrocarbons contribute in the degrading process of intermediate-degrading products thereof into carbon dioxide. Microorganisms that supply essential trace nutrients such as vitamins to these microorganisms are also believed to be present. It is conceivable that the petroleum is rendered inorganic and nontoxic through the cooperation of multiple types of microorganisms. When certain degradation products can serve as growth inhibitors, co-existing microorganisms which are capable of utilizing these degradation products in this case raise the degradation efficiency and actualize higher degradation function of the total microbial population.

Concretely speaking, the dominant level(s) of one or multiple types of microorganisms with specific functions within the microbial population, e.g., aerobic hydrocarbon-degrading bacteria or PCB-degrading bacteria, protein-degrading bacteria, glucose-assimilating bacteria, and anaerobic sulfate-reducing bacteria, and methanogenic bacteria, can be investigated by the aforementioned method. Investigation of the changes over time in the dominant level permits analysis of transitions in each of the microorganisms with specific functions. As a result, for example, if the dominant level of sulfate-reducing bacteria rises at a certain time in the coastal surface water, the latent sulfate-reducing function can be judged to be high in the microbial population at that time. Even before collecting samples, the cause of this can be hypothesized to be a change from the ordinary habitat due to factors such as upward rising of bottom mud due to a typhoon, or movement of the earth's crust, or influx of anaerobic wastewater, etc.

Polluted or contaminated environments can also be analyzed and evaluated using the aforementioned method. For example, when the subject microorganisms with specific functions are heterotrophs that are specifically dominant in pulp plant wastewater, elevation of their dominant level at a certain time can be judged to quite possibly be due to pollution of this environment by this wastewater. Gaining an understanding of the periodic and seasonal changes by long-term monitoring of the changes in the dominant level allow one to evaluate whether these changes are spontaneous or man-caused.

Environments polluted or contaminated by noxious chemicals can also be analyzed and evaluated using the aforementioned method. For example, when the subject microorganisms with specific functions are PCB-degrading bacteria, elevation of their dominant level at a certain time makes it possible to judge that there is a strong possibility that the environment has been polluted or contaminated by PCBs and the PCB-degrading function as the total microbial population can be judged to be elevated. Gaining an understanding of the periodic and seasonal changes by long-term monitoring of the changes in the dominant level allow one to evaluate whether the changes are spontaneous or man-caused as in influx of industrial waste water.

Oil-polluted environments can also be analyzed and evaluated by the aforementioned method. For example, when the microorganisms with specific functions of interest are bacteria that degrade petroleum components such as tetradecane and anthracene, elevation of their dominant levels at a certain time makes it possible to judge that there is a strong possibility that the environment has been polluted or contaminated by saturated hydrocarbons such as n-alkane among the petroleum components in the case of the former and by aromatic hydrocarbons in the case of the latter and that the petroleum component-degrading function as the total microbial population has been elevated. Investigating changes in their dominant levels can also make it possible to analyze and evaluate the extent and the degree of the oil pollution and the bioremediation process. Gaining an understanding of the periodic and seasonal changes by long-term monitoring of the changes in the dominant level allow one to evaluate whether the changes are spontaneous or man-caused as in a maritime accident.

When attention is turned to aliphatic hydrocarbon-degrading bacteria known to be widely distributed throughout the oceans as the microorganisms with specific functions, DNA and RNA probes which have a length of 10 to 50 bases, 15 to 25 bases, can be designed such that comprise all or part of the nucleotide sequence of SEQ ID NO:1 (which is the region of nucleotide numbers. 207 to 226 in SEQ ID NO:5; or the region of nucleotide numbers. 222 to 241 in the numbering system that shows the position from the 5′-end in the nucleotide sequence of Escherichia coli 16S rDNA (see, e.g., Noller (1981) Science 212:403-411), which have part of the nucleotide sequence (SEQ ID NO:5) of 16S rDNA of the aliphatic hydrocarbon-degrading bacterial strain GR211-P1 (Accession NO. FERM P-17394) isolated in purity by enrichment culture from the costal area of Shikoku, and which specifically hybridize with corresponding nucleic aid sequences from petroleum-degrading bacteria of the genus Alcanivorax, particularly Alcanivorax borkumensis and closely related species, to permit detection and determination of the petroleum-degrading bacterium. The following probe can be given as an example.

| | |
|---|---|
| (1) 5′-CGA CGC GAG CTC ATC CAT CA-3′ (Albo 222, 20 mer) | (SEQ ID NO: 1) |
| 3′-GCT GCG CTC GAG TAG GTA GT-5′ | (SEQ ID NO: 7) |

These probes should be the synthesized and non-RI-labeled, i.e., labeled with a fluorescent dye or tag, using the methods described above.

When attention is turned to aromatic hydrocarbon-degrading bacteria, the distribution of which has been confirmed in multiple sea areas, as the microorganisms with specific functions, DNA and RNA probes with the following nucleotide sequences can be used that were designed to detect aromatic hydrocarbon-degrading bacteria of the genus Cycloclasticus that have part of the 16S rDNA nucleotide sequence (SEQ ID NO: 6) derived from the petroleum-degrading bacteria discovered in samples collected on Jan. 15, 1997 along the coast of Mikuni-machi in Fukui-ken where a large oil spill had occurred when the ship's bow hit the bottom following an oil spill accident in the Japan Sea caused by the Russian tanker Nakhodka, particularly Cycloclasticus pugetii and its closely related species.

(1) 5′GGAAACCCGCCCAACAGT-3′(Cypug829-846*, 18mer: region of nucleotide numbers. 823 to 840 in SEQ ID NO: 6) (SEQ ID NO: 2) 3′-CCTTTGGGCGGGTTGTCA-5′ (SEQ ID NO: 8)

(2) 5′-TGCACCACTAAGCGGAAACC-3′(Cypug840-859*, 20 mer: region of nucleotide numbers. 834 to 853 in SEQ ID NO: 6) (SEQ ID NO: 3) 3′-ACGTGGTGATTCGCCTTTGG-5′ (SEQ ID NO: 9)

(3) 5′-TGCACCACTAAGCGGAAACCCGCCCAACAGT-3′(Cypug829-859*, 31 mer: region of nucleotide numbers. 823 to 853 in SEQ ID NO: 6) (SEQ ID NO: 4)

(The numbers with an asterisk show the position from the 5′-end in the Escherichia coli 16S rDNA sequence (numbering system); see, e.g., Noller (1981) Science 212:403-411).

Even though the nucleotide sequence of the probe (3) is adjacent to the nucleotide sequences of probes (1) and (2), attention has to be given in the case of this probe to the possibility of self-binding.

The respective dominant levels of aliphatic hydrocarbon-degrading bacteria of the genus Alcanivorax, especially Alcanivorax borkumensis and its closely related species, and aromatic hydrocarbon-degrading bacteria of the genus Cycloclasticus, especially Cycloclasticus pugetii and its closely related species, among the total microbial population and/or in the Bacteria domain to which the two belong can be measured accurately at the molecular level by applying the aforementioned molecular genetic detection and quantification technique (relative molecular quantification method) to the subject environmental microorganism-containing samples using DNA probes for the detection of the domains to which the microorganisms with specific functions belong, and universal probes for all living organisms including these, in addition to the above DNA probes for detection of these microorganisms with specific functions. Furthermore, analyzing temporal and/or spatial changes in this dominant level makes it possible to analyze and diagnose from the microbiological viewpoint the state of natural environments and artificial environments, particularly oil-spill environments that have been polluted by petroleum.

The method of the present invention makes it possible to diagnose simply and rapidly environments that have been polluted by noxious chemicals such as petroleum by a molecular genetic technique that does not depend on conventional isolation and culture methods and does not use RI (radioisotopes) or PCR by taking as the subjects microorganisms that live in that environment. The method of the present invention may be also applied for detection, quantification, and screening of microorganisms with specific functions, particularly microorganisms producing useful substances such as useful enzymes and antibiotics. Furthermore, the method of the present invention may be applied to detection, quantification, and screening of harmful microorganisms including pathogenic microbes. Therefore, the present invention makes it possible to design kits and automation and is extremely advantageous as a diagnostic tool for the environments that have been polluted by noxious chemicals and for analyzing and evaluating the repair effects of the bioremediation process. In addition, the present invention is also useful for detection, quantification, and screening of microorganisms with specific functions, including microorganisms producing useful substances and harmful microorganisms such as pathogenic microbes, contained in a natural or artificial environment. Thus, the present invention can also be useful for analysis and evaluation of environmental samples containing the microorganisms with specific functions.

The DNA probes presented in the present invention are specifically distinguishable for specific aliphatic hydrocarbon-degrading bacteria that have been reported to appear in many sea areas in recent years and are extremely advantageous for their detection and quantification.

The present invention provides a method of analyzing the dominant level of microorganisms with specific function (for example, petroleum-degrading bacteria, microorganisms producing beneficial substances, pathogenic microorganisms, and sulfate-reducing bacteria) existing in natural environments and artificial environments (such as petroleum-polluted environments) with the molecular genetic technique without using a conventional culture and counting method which requires so significant time and effort. Furthermore, the present method is an advantageous non-RI and non-PCR method, which does not require any radioisotope and PCR procedure.

The nucleotide sequence of the novel nucleic acid probe specific for the petroleum-degrading bacterium Alcanivorax borkumensis is disclosed herein, which was obtained according to the method of the present invention. The bacterium is one of the natural petroleum-degrading microorganisms, and is possible to dominate widely in oil-spill shoreline environments. Therefore, the sequence of this probe is extremely useful for analyzing the behavior of these petroleum-degrading microorganisms in nature, under petroleum treatment conditions such as the process of bioremediation of oil-polluted environments, under experimental conditions in validation test of bioremediation techniques in the open air, or under experimental conditions in laboratories. The nucleotide sequence of the novel nucleic acid probe is also useful as a diagnostic tool for oil-polluted environments, and for evaluating repair efficacy of bioremediation in oil-polluted environments.

The present invention demonstrated the dominant level analysis of representative aliphatic hydrocarbon-degrading bacteria and/or representative aromatic hydrocarbon-degrading bacteria, which are known to be widely distributed along shorelines, by a molecular genetic technique. Therefore, the present invention is proved to be useful for biologically monitoring the state of residues and degrading process of petroleum components in petroleum-polluted seas. Further the present invention may be very effective and useful for developing environmental repair techniques (bioremediation techniques) to stimulate microbial degradation of petroleum in oil-polluted environments by adding inorganic nutrients and/or organic compounds, such as surfactants and growth promoting factors, along with/without petroleum-degrading microorganisms. Furthermore, the present invention is extremely advantageous for detection, quantification, and screening of microorganisms with specific functions including microorganisms producing useful substances and harmful microorganisms such as a pathogenic microbe, and for analysis and evaluation of environmental samples containing the microorganisms with specific functions.

It will be readily apparent to one skilled in the art that various substitutions and modifications may be made to the invention disclosed herein without departing from the scope and spirit of the invention. It is understood that the examples and aspects described herein are for illustrative purposes only and that various modifications or changes in light thereof will be suggested to persons skilled in the art and are to be included within the spirit and purview of this application and scope of the appended claims.

### EXAMPLES

The following examples are offered to illustrate, but not to limit the claimed invention.

### Example 1

Development of Probes for the Detection of Alcanivorax and Cycloclasticus

(1) Development of a Nucleic Acid Probe for the Detection of Alcanivorax borkumensis

To develop a DNA probe for detection of Alcanivorax borkumensis, a sequence unique to the aliphatic hydrocarbon-degrading bacterial strain GR211-P1 (Accession NO:FERM P-17394), which has been successfully isolated through enrichment cultivation of a sample from the costal area of Shikoku (Japan), was selected by retrieval for the region that does not exhibit damage due to higher structures shown by Stahl and Amann, Development and Application of Nucleic Acid Probes, In: Nucleic Acid Techniques in Bacterial Systematics, Ed.: E. Stackebrandt and M. Goodfellow, John Wiley and Sons, Chichester, pp. 205-248, 1991; from among the 16S rDNA ribonucleotide sequence information (SEQ ID NO: 5) of the bacterial strain GR211-P1. Then the nucleotide having the selected sequence presented by SEQ ID NO:1 was synthesized with an OLIGO 1000M™ system (Beckman Coulter, Tokyo, Japan). The resulted nucleotide was labeled with FITC, TRITC, or Cy5 fluorescent dye on its 5′-end, thus a non-RI-labeled DNA probe comprising sequence NO:1 was obtained (which sometimes referred to hereinafter as Albo 222).

By analyzing the sequence of this probe in database, it was confirmed that this probe exhibits 100% homology to only Alcanivorax borkumensis SK2 (type strain; M. M. Yakimov et al.: 1998, ibid.) and its closely related strain Fundibacter jadensis T9 (type strain). This showed it to be advantageously useful as a DNA probe for detection of Alcanivorax borkumensis and the phylogenetic group of Alcanivorax. Since the closely related species Fundibacter jadensis (Bruns (1999): Fundibacter jadensis gen. nov., sp. nov., a new slightly halophilic bacterium, isolated from intertidal sediment, Int. J. Syst. Bacteriol., 49:441-448) is regarded to belong to the phylogenetic group identical to Alcanivorax borkumensis in molecular phylogeny. Furthermore, the species has high homology (approximately 98%) of the full-length nucleotide sequence. From these knowledge, it was judged to be a species closely related or identical to Alcanivorax borkumensis quite possibly included in the genus Alcanivorax or the same species.

The utility of the DNA probe of the present invention was confirmed by FISH method using Alcanivorax borkumensis DSM11573 (type strain) as the target microorganism and Pseudomonas aeruginosa IFO12689 (type strain) as the control microorganism with the apparatus and procedures described in Maruyama (2000) Simultaneous direct counting of total and specific microorganism cells in seawater, using a deep-sea microorganism as biomarker, Applied and Environmental Microbiology 66:2211-2215). Hybridization in this case was performed at 43° C. in the presence of 20% formamide, and washing was performed at 44° C.

Preparation of DNA Probe for Detection of Cycloclasticus pugetii

As the DNA probe for detection of another target microorganism, Cycloclasticus pugetii, Cyclopug829-846 (sequence NO: 2: sometimes referred to hereinafter as Cypug829) was used. This probe sequence was discovered from the 16S rDNA nucleotide sequence determined using DNAs which were extracted directly and readily from sample of growth-positive front well used in MPN counting for petroleum-degrading bacteria that employed only heavy fuel oil as the carbon source from samples collected on Jan. 15, 1997 from coastal area of Mikuni-machi in Fukui-ken, Japan, where after an oil spill accident in the Japan Sea caused by the Russian tanker Nakhodka on Jan. 2, 1997, a large oil spill occurred when the bow of the tanker ran aground a reef off of the coast.

### Example 2

Analyzing a Dominant Level of a Microorganism with a Specific Function in a Selected Environment

The following results were obtained by analysis of dominant levels of Cycloclasticus pugetii and Alcanivorax borkumensis using DNA probes mentioned above in a actual petroleum-polluted sea area.

1) Collection and Storage of Samples

Samples were cryopreserved until used after collection on Jan. 15, 1997, from coastal area of Mikuni-machi in Fukui-ken, Japan, where a large oil spill occurred described above. Specifically, approximately 3 L of collected oil-polluted seawater were concentrated on a cartridge-type hollow-fiber filter HF400™ (Millipore, Bedford, Mass.) with a pore size of 0.2 μm by filtering, and then the sample with the cartridge filter was immediately frozen and stored.

2) Cell Disruption, and Extraction and Storage of RNA

The concentrated microbial samples in the cartridge from the environment were transferred to a 12 mL hard glass test tube using the RLT buffer provided in the RNEASY MIDI KIT™ (Qiagen, Tokyo, Japan). After adding 1.5 g of glass beads (GLASS BEADS 106™ microns and fmer; Sigma, St. Louis, Mo.) per 1.5 mL of the used RLT buffer, which had previously been subjected to dry heat sterilization for at least 1 hour at 180° C., the microorganism cells were disrupted by reciprocal shaking for 5 minutes with a CELL HOMOGENIZER MSK™ (B. Braun Biotech International, Melsungen, Germany). The homogenizing treatment was conducted by keeping approximately at 5° C. while monitoring with electronic thermometer and spraying liquid carbon dioxide. The treatment time was decided as the treatment condition that may extract a high quantity of nucleic acid without excessive fragmentation thereof. For deciding this condition, after homogenizing treatment of Bacillus subtilis IFO13719 cells, a kind of Gram-positive bacteria which have harder cell walls and are more common on land than Gram-negative bacteria, the eluted nucleic acid was analyzed by agarose gel electrophoresis, and the degree of cell disruption was observed under a microscope. The RNA was extracted and purified from the resulted cell-disrupted samples using an RNEASY MIDI KIT™ (Qiagen), and cryopreserved at −80° C. after aliquotted into from 50 to 100 μL portions.

3) Measurement of Amount of Total RNA

Prior to hybridization analysis, the total amount of RNA contained in the sample was measured with a RIBOGREEN RNA QUANTITATION KIT™ (Molecular Probes, Eugene, Oreg.) and fluorescence spectrophotometer RF-5300PC™ (Shimadzu Co. Kyoto, Japan) at an excitation wavelength of 480 nm, emission wavelength of 520 nm, and bandwidth of 5 nm.

4) Hybridization by Non-RI Method

Hybridization by non-RI method using the fluorescent probes was performed as follows. The cryopreserved sample was dissolved by adding the buffered solution prepared by mixing of a solution of pyrocarbonate (DMPC)-treated water, 20×SSC (0.15 M NaCl and 0.015 M sodium citrate), and formaldehyde, at the ratio of 5:3:2. The nucleic acid in the sample was blotted to a positively charged nylon membrane filter (Roche Diagnostics, Mannheim, Germany) and immobilized to the filter using a UV STRATALINKER™ (Stratagene). Prehybridization on the filter was conducted for 30 minutes at 35° C. with hybridization solution (Raskin (1994): Group-Specific 16S rRNA Hybridization Probes to Describe Natural Communities of Methanogens, Applied and Environmental Microbiology 60:1232-1240). Then, hybridization was performed using the hybridization solution identical to above containing each of Cy5-labeled DNA probe for detection of Alcanivorax borkumensis or Cycloclasticus pugetii at a final concentration of 10 pmol/mL, overnight at 35° C. The filter was washed in wash solution (1×SSC [0.15M NaCl and 0.015M sodium citrate] with 1% sodium dodecyl sulfate added) twice for 30 minutes each at the washing temperature optimum for each probe, which is determined by follows.

5) Determination of Probe Dissociation Calibration Curve by Non-RI Method

The aforementioned optimum washing temperature was determined by non-RI method based on the method described by Zheng et al. (Zheng (1996) ibid.). Specifically, the sample of extracted nucleic acid from each type strain (Cycloclasticus pugetii ATCC51542 and Alcanivorax borkumensis DSM11573) was dot-blotted (using approximately 2 μg of RNA per dot) onto a positively charged nylon membrane (Roch Diagnostics, Manheim, Germany), and then the membrane was subjected to hybridization with each DNA probe by the above method overnight at 35° C. The membrane was subsequently cut into strips with eight dots on each. The strips were transferred into 5 mL plastic test tubes containing 2 mL of wash solution. Next, the strips were washed at temperature raised by 3° C. from 35 to 80° C., wherein the fresh wash solution was used for each washing step at each temperature. The fluorescence from the probe dissociated from the nucleic acid on the membrane into each wash solution was measured by fluorescence spectrophotometer RF-5300PC™ (Shimadzu, Kyoto, Japan) at excitation wavelength of 640 nm, fluorescence wavelength of 662 nm, and bandwidth of 5 nm. Finally, the probe-wash-off curve for each probe was determined from the measured level of fluorescence. The optimum wash temperature was determined to be 47° C. for the probe for detection of Alcanivorax borkumensis and 41° C. for the probe for Cycloclasticus pugetii.

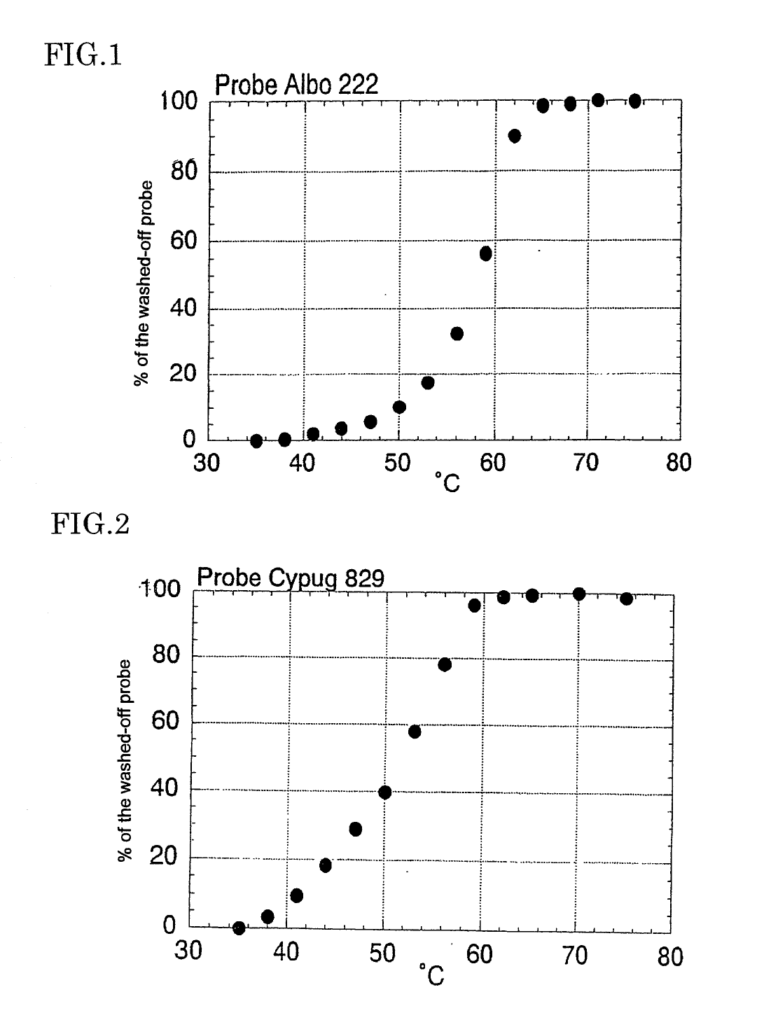

FIG. 1 and FIG. 2 show determined probe-wash-off curves for each probe for detection of Alcanivorax borkumensis (Albo222and of Cycloclasticus pugetii (Cypug829) respectively.

6) Obtaining Fluorescent Images and Quantitative Analysis Thereof

Using the above filters that had been hybridized with the Cy5-labeled DNA probes and, fluorescent dot images of the fluorescence from DNA probes hybridized to nucleic acid on the filters were obtained using a fluorescent image analyzer STORM™ (Amersham Pharmacia Biotech)by excitation with red laser diode (output 5 mW, wavelength 635 nm) and a corresponding fluorescence filter (650 nm long-pass filter). The quantified values were then determined by analyzing the fluorescent images using an IPLAB™ (Scanalytics, Fairfax, Va.). Specifically, the real quantified value was determined by subtracting the background value from the image of tested sample, and by the use of a calibration curve determined from the images of stepwise-diluted samples of E. coli 16S rRNA standard samples blotted on the same membrane.

7) Dominant Level Analysis of Specific Microorganisms

The dominant level of the target microorganisms to all living organisms and each bacterial domain was calculated by the above formula 1 advocated by an RI method.

(Correction of Determined Values)

Here, the values used in calculation of dominant level of each domain were determined with the following standard strains.

Bacteria domain

Escherichia coli DSM30083

Psychrobacter pacificensis IFO16279

Cycloclasticus pugetii ATCC51542

Alcanivorax borkumensis DSM11573

Arcobacter nitrofigilis DSM7299

Methylobacterium extorquens DSM1053

Methylophilus mthylotrophus DSM46235

Archaea domain

Aeropyrum pernix JCM9820

Methanococcoides methylutens DSM2657

Eucarva domain

Saccharomyces cerevisiae IFO10217

From the type strains used in correction, nucleic acid was extracted with RNEASY MAXI KIT™ (Qiagen), aliquoted into 200 μL portions, and stored at −80° C.

Using the stored nucleic acid samples, each of the procedures of determination of total RNA amount, hybridization and washing, obtaining dot images, and quantitative image analysis was performed as described above.

As probes for hybridization for each domain, Euba 338 (Bacteria domain), Euka 502 (Eucarya domain), and Arch 915 (Archaea domain) described by Amann et al. (Amann (1995) ibid.) were used. As the DNA probe for the universal region of all living organisms, Univl390 (Zheng (1996) ibid.) was used.

The above species-specific DNA probes Cypug829 and Albo222 were used for the target microorganisms with a specific function, Cycloclasticus pugetii and Alcanivorax borkumensis in the Bacteria domain respectively.

The results of the analysis are shown in Table 1. In Table 1, sample #1 was collected at the Anto fishing port of Mikuni-machi (Stn. A), and sample #2 was collected at a reef near where after the Russian tanker's accident the bow of the ship hit bottom on the north shore of Anto near the same town (Stn. B).

TABLE 1

| | | Sample #1: Dominant | Sample #2: Dominant |
|---|---|---|---|
| Domain | Probe | Level (%) | Level (%) |
| Bacteria | Euba338 (total | 45.2 (100) | 53.3 (100) |
| | amount) | | |
| | Cypug829 | 10.7 (23.6) | 13.1 (24.5) |
| | Albo222 | 2.0 (4.4) | 3.5 (6.5) |
| Eukaria | Euka502 | 19.9 | 24.0 |
| Archia | Arch915 | 8.5 | 13.3 |
| Total | | 73.6 | 90.6 |

As shown in Table 1, approximately 45.2% (61.4% taking the whole as 100%) of Bacteria domain, approximately 19.9% of Eucarya domain, and approximately 8.5% Archaea domain of (total 73.6%) were detected in sample 1. Approximately 53.3% (59% taking the whole as 100%) Bacteria domain, approximately 24.0% Eucarya domain, and approximately 13.3% Archaea domain (total 90.6%) were detected in sample 2.

The numbers in parentheses under the Bacteria domain in Table 1 show the percentages of the values for Cypug829 and Albo222 taking the value for Euba338, which express the dominant level of the whole domain, as 100%. Specifically, in the Bacteria domain in sample 1, the dominant level of the aromatic hydrocarbon-degrading bacterium Cycloclasticus pugetii, a target microorganism that belongs to this domain, is 23.6% and that of the aliphatic hydrocarbon-degrading bacterium Alcanivorax borkumensis, another target microorganism, is 4.4%. Similarly, in the Bacteria domain in sample 2, the dominant level of the aromatic hydrocarbon-degrading bacterium Cycloclasticus pugetii is 24.5% and that of the aliphatic hydrocarbon-degrading bacterium Alcanivorax borkumensis is 6.5%.

These results proved that the dominant levels of microorganisms with specific functions determined by a non-RI method described above using nucleic acids samples extracted from the microorganisms collected from polluted environments are enable molecular-genetic analysis, evaluation, and diagnosis of actual polluted environments.

A number of embodiments of the invention have been described. Nevertheless, it will be understood that various modifications may be made without departing from the spirit and scope of the invention. Accordingly, other embodiments are within the scope of the following claims.

What is claimed is:

1. A method for analyzing a dominant level of a microorganism in an environment, wherein the microorganism comprises a specific function, by detecting and quantifying nucleic acids of the microorganism from a natural or an artificial environment by a non-radioisotope (RI) method comprising the following steps:
(a) extracting, purifying, and preserving a target nucleic acid from a microorganism-containing sample collected from a natural or an artificial environment;
(b) selecting a specific nucleic acid sequence from sequence information of the target nucleic acid of the microorganism, and synthesizing a nucleic acid with the selected sequence, and labeling the synthesized nucleic acid with a non-RI-label to prepare a non-RI-labeled nucleic acid probe specific for the microorganism;
(c) immobilizing the target nucleic acid extracted and purified in step (a) to a substrate for hybridization, and adding the non-RI-labeled nucleic acid probe specific for said microorganism; followed by hybridization and washing;
(d) obtaining an image of the signals derived from a non-RI label specifically hybridized to an immobilized nucleic acid probe, and determining from the image quantified values of the target nucleic acid from said microorganism; and
(e) calculating a dominant level of the microorganism by comparing the quantified values of the microorganism in step (d) with those determined for all organisms or domains in the living world.

2. The method of claim 1 wherein the washing of step (c) is performed at an optimum washing temperature which is determined from the probe-wash-off curve for the non-RI-labeled nucleic acid probe specific for the microorganism obtained by a non-RI method.

3. The method of claim 1 wherein the non-RI-labeled nucleic acid probe is labeled with a fluorescent or a chemiluminescent label.

4. The method of claim 1, wherein the microorganism with a specific function is a microorganism that degrades a specific chemical.

5. The method of claim 4 wherein the specific chemical comprises a noxious chemical.

6. The method of claim 4 wherein the specific chemical comprises a petroleum or a petroleum component.

7. The method of claim 4 wherein the specific chemical comprises a PCB.

8. The method of claim 1, wherein the microorganism with a specific function is a microorganism that produces a useful substance.

9. The method of claim 8, wherein the useful substance is an enzyme.

10. The method of claim 1, wherein the microorganism with a specific function is a harmful microorganism including pathogenic microbe.

11. The method of claim 1, wherein the nucleic acid is an RNA or a DNA.

12. The method of claim 1, wherein the nucleic acid probe has a length of between about 10 to about 50 bases, between about 15 to about 40 bases, between about 20 to about 30 bases, or between about 15 to about 25 bases.

13. A method for evaluating and diagnosing a function of a microbial population in an enviromnent, wherein the method comprises analyzing a dominant level of a microorganism with a specific function in a natural or an artificial environment using a method comprising the following steps:
(a) extracting, purifying, and preserving a target nucleic acid from a microorganism-containing sample collected from the natural or the artificial environment;
(b) selecting a specific nucleic acid sequence from sequence information of the target nucleic acid of a microorganism, and synthesizing a nucleic acid with the selected sequence, and labeling the synthesized nucleic acid with a non-radioisotope (RI)-label to prepare a non-RI-labeled nucleic acid probe specific for the microorganism;
(c) immobilizing the target nucleic acid extracted and purified in step (a) to a substrate for hybridization, and adding the non-RI-labeled nucleic acid probe specific for the microorganism, followed by hybridization and washing;
(d) obtaining an image of the signals derived from a non-RI label specifically hybridized to an immobilized nucleic acid probe, and determining from the image quantified values of the target nucleic acid from the microorganism; and
(e) calculating a dominant level of the microorganism through comparing the quantified values of the microorganism in step (d) with those determined for all organisms or domains in the living world.

14. A method for analyzing and diagnosing a polluted or contaminated environment using a method comprising the following steps:
(a) extracting, purifying, and preserving a target nucleic acid from a microorganism-containing sample collected from a natural or an artificial environment;
(b) selecting a specific nucleic acid sequence from sequence information of the target nucleic acid of a microorganism, wherein the microorganism has a specific function, and synthesizing a nucleic acid with the selected sequence, and labeling the synthesized nucleic acid with a non- radioisotope (RI)-label to prepare a non-RI-labeled nucleic acid probe specific for the microorganism;
(c) immobilizing the target nucleic acid extracted and purified in step (a) to a substrate for hybridization, and adding the non-RI-labeled nucleic acid probe specific for the microorganism, followed by hybridization and washing;
(d) obtaining an image of the signals derived from a non-RI label specifically hybridized to an immobilized nucleic acid probe, and determining from the image, quantified values of the target nucleic acid from the microorganism; and
(e) calculating a dominant level of the microorganism through comparing the quantified values of the microorganism in step (d) with those determined for all organisms or domains in the living world.

15. A method for analyzing and diagnosing an environment polluted or contaminated by a noxious chemical, comprising the following steps:
(a) extracting, purifying, and preserving a target nucleic acid from a microorganism-containing sample collected from a natural or an artificial environment;
(b) selecting a specific nucleic acid sequence from sequence information of the target nucleic acid of a microorganism, wherein the microorganism is capable of degrading a specific chemical, and synthesizing a nucleic acid with the selected sequence, and labeling the synthesized nucleic acid with a non-radioisotope (RI)-label to prepare a non-RI-labeled nucleic acid probe specific for the microorganism;
(c) immobilizing the target nucleic acid extracted and purified in step (a) to a substrate for hybridization, and adding the non-RI-labeled nucleic acid probe specific for the microorganism, followed by hybridization and washing;
(d) obtaining an image of the signals derived from a non-RI label specifically hybridized to an immobilized nucleic acid probe, and determining from the image, quantified values of the target nucleic acid from the microorganism; and
(e) calculating a dominant level of the microorganism through comparing the quantified values of the microorganism in step (d) with those determined for all organisms or domains in the living world.

16. A method for analyzing and diagnosing an oil-polluted or contaminated environment comprising the following steps:
(a) extracting, purifying, and preserving a target nucleic acid from a microorganism-containing sample collected from a natural or an artificial environment;
(b) selecting a specific nucleic acid sequence from sequence information of the target nucleic acid of a microorganism, wherein the microorganism is capable of degrading a specific chemical and the specific chemical comprises a petroleum or a petroleum component, and synthesizing a nucleic acid with the selected sequence, and labeling the synthesized nucleic acid with a non-radioisotope (RI)-label to prepare a non-RI-labeled nucleic acid probe specific for the microorganism;
(c) immobilizing the target nucleic acid extracted and purified in step (a) to a substrate for hybridization, and adding the non-RI-labeled nucleic acid probe specific for the microorganism, followed by hybridization and washing;
(d) obtaining an image of the signals derived from a non-RI label specifically hybridized to an immobilized nucleic acid probe, and determining from the image, quantified values of the target nucleic acid from the microorganism; and
(e) calculating a dominant level of the microorganism through comparing the quantified values of the microorganism in step (d) with those determined for all organisms or domains in the living world.

17. A nucleic acid probe having a length of between about 10 to about 50 bases comprising any part of the nucleotide sequence of SEQ ID NO:5, or a corresponding RNA sequence thereto, wherein the probe is capable of hybridizing specifically with a nucleic acid derived from any petroleum-degrading bacterium belonging to the phylogenetic group or genus of Alcanivorax.

18. The nucleic acid probe of claim 17, wherein the nucleic acid is an RNA or a DNA.

19. The nucleic acid probe of claim 17, wherein the nucleic acid probe has a length of between about 15 to about 40 bases, between about 20 to about 30 bases, or between about 15 to about 25 bases.

20. A nucleic acid probe comprising a nucleotide sequence as set forth in SEQ ID NO: 1, or a corresponding RNA sequence thereto, wherein the probe is capable of detecting or quantifying a petroleum-degrading bacterium by hybridizing specifically with a nucleic acid derived from any petroleum-degrading bacterium belonging to the phylogenetic group or genus of Alcanivorax.

21. The nucleic acid probe of claim 20, wherein the nucleic acid is an RNA or a DNA.

22. The nucleic acid probe of claim 20, wherein the nucleic acid probe has a length of between about 10 to about 50 bases, between about 15 to about 40 bases, between about 20 to about 30 bases, or between about 15 to about 25 bases.

23. The nucleic acid probe of claim 17 or claim 20, wherein the petroleum-degrading bacteria belonging to the phylogenetic group or genus of Alcanivorax comprise Alcanivorax borkumensis or its closely related species.

24. A method for analyzing and diagnosing an oil-polluted or contaminated environment, wherein the method comprises analyzing a dominant level of a petroleum-degrading bacterium belonging to the phylogenetic group or genus of Alcanivorax and/or to the genus of Cycloclasticus in microbial population in the environment by a method comprising the following steps:
(a) extracting, purifying, and preserving a target nucleic acid from a microorganisms-containing sample collected from the natural or artificial environment;
(b) selecting a specific nucleic acid sequence from sequence information of the target nucleic acid of the microorganism with a specific function, and synthesizing a nucleic acid with the selected sequence, and labeling the synthesized nucleic acid with a non-radioisotope (RI)-label to prepare a non-RI-labeled nucleic acid probe specific for the microorganism;
(c) immobilizing the target nucleic acid extracted and purified in step (a) to a substrate for hybridization, and adding the non-RI-labeled nucleic acid probe specific for the microorganism, followed by hybridization and washing;
(d) obtaining an image of the signals derived from a non-RI label specifically hybridized to an immobilized nucleic acid probe, and determining from the image, quantified values of the target nucleic acid from the microorganism; and
(e) calculating a dominant level of the microorganism through comparing the quantified values of the microorganism in step (d) with those determined for all organisms or domains in the living world;
wherein the method uses as a non-RI-labeled nucleic acid probe specific for a certain microorganism with a specific function at least one probe selected from the group consisting of: a nucleic acid probe having a length of about 10 to about 50 bases comprising any part of the nucleotide sequence of SEQ ID NO:5, or a corresponding RNA sequence thereto, wherein the probe is capable of hybridizing specifically with a nucleic acid derived from any petroleum-degrading bacterium belonging to the phylogenetic group or genus of Alcanivorax; a nucleic acid probe comprising a nucleotide sequence as set forth in SEQ ID NO:1, or a corresponding ribonucleotide sequence thereto, wherein the probe is capable of hybridizing specifically with nucleic acids derived from any of petroleum-degrading bacteria belonging to the phylogenetic group or genus of Alcanivorax; and, a probe having length of from about 10 to about 50 bases comprising any part of a nucleotide sequence as set forth in SEQ ID NO:6 or a corresponding ribonucleotide sequence thereto; and, a DNA probe comprising a sequence as set forth in SEQ ID NOs:2-4, or corresponding RNA probes thereto, which probes are capable of hybridizing specifically with a nucleic acid derived from any of petroleum-degrading bacteria belonging to the genus of Cycloclasticus.

25. The method of claim 24, characterized by analyzing a dominant level of any microorganism selected from the group consisting of an aliphatic hydrocarbon-degrading bacterium, Alcanivorax borkumensis, or its closely related species and/or an aromatic hydrocarbon-degrading bacterium, Cycloclasticus pugetii, or its closely related species in the microbial population in an environment.

26. An environmental diagnostic kit comprising a non-RI-labeled probe, wherein the probe is prepared by non-radioisotope (RI)-labeling a nucleic acid probe and the probe is selected from the group consisting of: a nucleic acid probe having a length of between about 10 to about 50 bases comprising any part of the nucleotide sequence of SEQ ID NO:5, or a corresponding RNA sequence thereto, wherein the probe is capable of hybridizing specifically with a nucleic acid derived from any petroleum-degrading bacterium belonging to the phylogenetic group or genus of Alcanivorax; and, a nucleic acid probe having length of between about 10 to about 50 bases containing any part of the nucleotide sequence SEQ of ID NO:6 or a corresponding ribonucleotide sequence thereto which is capable of hybridizing specifically with a nucleic acid derived from a petroleum-degrading bacterium belonging to the genus Cycloclasticus; and, a nucleic acid probe having a nucleotide sequence as set forth in SEQ ID NOs:1-4 or corresponding ribonucleotide sequences thereto.

27. The environmental diagnostic kit of claim 26, wherein the nucleic acid is an RNA or a DNA.

28. The environmental diagnostic kit of claim 26, wherein the nucleic acid probe has a length of between about 15 to about 40 bases, between about 20 to about 30 bases, or between about 15 to about 25 bases.

29. The environmental diagnostic kit of claim 26, wherein the petroleum-degrading bacteria belonging to the phylogenetic group or genus of Alcanivorax comprise Alcanivorax borkumensis or its closely related species.


SEQUENCE LISTING (9 sequences)

SEQ ID NO: 1 - DNA, 20 bp, Artificial Sequence
  Probe
cgacgcgagctcatccatca

SEQ ID NO: 2 - DNA, 18 bp, Artificial Sequence
  Probe
ggaaacccgcccaacagt

SEQ ID NO: 3 - DNA, 20 bp, Artificial Sequence
  Probe
tgcaccactaagcggaaacc

SEQ ID NO: 4 - DNA, 31 bp, Artificial Sequence
  Probe
tgcaccactaagcggaaacccgcccaacagt

SEQ ID NO: 5 - DNA, 1494 bp, Unknown
  GR211-P1(FERM P-17394)
ttatcatggctcagattgaacgctggcggcaggcctaacacatgcaagtcgagcggaaacgatcctagcttgctaggaggcgtcgagcggcggacgggtgagtaacacgtgagaatctgcccattagagggggataacctggggaaacccaggctaataccgcataatccctacgggggaaagcaggggatcttcggaccttgtgctgatggatgagctcgcgtcggattagcttgttggtgaggtaatggctcaccaaggcgacgatccgtagctggtcttagaggatgatcagccacaccgggactgagacacggcccggactcctacgggaggcagcagtggggaatcttggacaatgggggcaaccctgatccagccatgccgcgtgtgtgaagaaggccttcgggttgtaaagcactttcagtagggaggaaggcttatccttaatacggatgagtacttgacgttacctacagaagaagcaccggctaatttcgtgccagcagccgcggtaatacgaaaggtgcgagcgttaatcggaattactgggcgtaaagcgcgcgtaggcggtttgctaagtcagatgtgaaagccccgggctcaacctgggaactgcatttgaaactggcaggctagaatgcagtagagggaggtggaatttccggtgtagcggtgaaatgcgtagagatcggaaggaacaccagtggcgaaggcggcctcctggactgacattgacgctgaggtgcgaaagcgtggggagcaaacaggattagataccctggtagtccacgccgtaaacgatgtctactagtcgttgggaatcttagtattcttggtgacgaagttaacgcgataagtagaccgcctggggagtacggccgcaaggttaaaactcaaatgaattgacgggggcccgcacaagcggtggagcatgtggtttaattcgatgcaacgcgaagaaccttaccaggccttgacatccttggaactttctagagatagattggtgccttcgggagccaagtgacaggtgctgcatggctgtcgtcagctcgtgtcgtgagatgttgggttaagtcccgtaacgagcgcaacccttgtccctagttgccagcacgtaatggtgggaactctagggagactgccggtgacaaaccggaggaaggtggggacgacgtcaagtcatcatggcccttacggcctgggctacacacgtgctacaatgggcagtacagagggcagcaaagtcgcgaggccaagcaaatcccttaaaactgttcgtagtccggattggagtctgcaactcgactccatgaagtcggaatcgctagtaatcgcggatcagaatgccgcggtgaatacgttcccgggccttgtacacaccgcccgtcacaccatgggagtggattgcaccagaagtggatagtctaaccttcgggaggacgttcaccacggtgtggttcatgactggggtgaagtcgtaacaaggta

SEQ ID NO: 6 - DNA, 1532 bp, Cycloclasticus pugetii
agagtttgatcatggctcagattgaacgctggcggcatgcctaacacatgcaagtcgaacggaaacagaatgcagcttgctagcaggcggtcgagtggcggacgggtgagttatgcataggaatccgcccgatagtgggggacaacctcctgaaaacgctgctaataccgcataatcccgcgggggcaaagacggggaccttcgggccttgctctaatggatgagcctacaggggattaggtagttggtgaggtaacggctcaccaaggcaacgatccctagctggtttgagaggatgatcagccacactgggactgagacacggcccagactcctacgggaggcagcagtggggaatattgcacaatggaggaaactctgatgcagcaatgccgcgtgtgtgaagaaggccttagggttgtaaagcactttcagtagggaggaaaagtttaagggtaataacccttaggccctgacgttacctacagaagaagcaccggctaactccgtgccagcagccgcggtaatacggagggtgcaagcgttaatcggaattactgggcgtaaagcgcgcgcaggcggttaaacaagtcagatgtgaaagccccgggctcaacctgggaactgcatttgaaactggctagctagagtgtggtagaggagagtggaatttcaggtgtagcggtgaaatgcgtagatatctgaaggaacaccagtggcgaaggcggctctctggaccaacactgacgctgaggtgcgaaagcgtgggtagcaaacgggattagataccccggtagtccacgccgtaaacgatgtcaactaactgttgggcgggtttccgcttagtggtgcastaacgcaataagttgaccgcctggggagtacggccgcaaggctaaaactcaaatgaattgacgggggcccgcacaagcggtggagcatgtggtttaattcgatgcaacgcgaagaaccttacctacccttgacatacagagaactttctagagatagattggtgccttcgggaactctgatacaggtgctgcatggctgtcgtcagctcgtgtcgtgagatgttgggttaagtcccgtaacgagcgcaacccttatccttagttgctaccatttagttgggcactctaaggagactgccggtgataaaccggaggaaggtggggacgacgtcaagtcatcatggcccttatgggtagggctacacacgtgctacaatggccggtacagagggccgcaaactcgcgagagtaagctaatcccttaaagccggtcctagtccggattgcagtctgcaactcgactgcatgaagctggaatcgctagtaatcgcggatcagaatgccgcggtgaattcgttcccgggccttgtacacaccgcccgtcacaccatgggagtgggttgcaaaagaagtgggtaggctaaccttcgggaggccgctcaccactttgtgattcatgactggggtgaagtcgtaacaaggtagccctaggggaacctggggctggatcacctcctt

SEQ ID NO: 7 - DNA, 20 bp, Artificial Sequence
  Probe
gctgcgctcgagtaggtagt

SEQ ID NO: 8 - DNA, 18 bp, Artificial Sequence
  Probe
cctttgggcgggttgtca

SEQ ID NO: 9 - DNA, 20 bp, Artificial Sequence
  Probe
acgtggtgattcgcctttgg